(19) United States
(12) Patent Application Publication
(10) Pub. No.: US 2016/0010094 A1
(43) Pub. Date: Jan. 14, 2016

(54) TRANSGENIC CELL SELECTION

(71) Applicants: UNIVERSITE DE GENEVE, Geneve 4 (CH); UNIVERSITY OF PRETORIA, Pretoria (ZA)

(72) Inventors: Karl-Heinz Krause, Geneve 4 (CH); David Suter, Geneve 4 (CH); Patrick Salmon, Geneve 4 (CH); Renier Myburgh, Pretoria (ZA); Michael Pepper, Pretoria (ZA)

(73) Assignees: UNIVERSITY OF PRETORIA, Pretoria (ZA); UNIVERSITE DE GENEVE, Geneve 4 (CH)

(21) Appl. No.: 14/771,599

(22) PCT Filed: Feb. 28, 2014
(86) PCT No.: PCT/IB2014/000994

§ 371 Date: Aug. 31, 2015

Related U.S. Application Data
(60) Provisional application No. 61/771,331, filed on Mar. 1, 2013.

(51) Int. Cl. C12N 15/65; A61N 5/10; A61K 31/17; A61K 48/00; A61K 31/517

(52) U.S. Cl. CPC C12N 15/65; A61K 48/00; A61K 31/517; A61K 31/17; A61N 5/1084

(57) ABSTRACT

Methods for selecting transgenic cells comprising two or more drug resistance genes with a combination of cytotoxic drugs (e.g., trimetrexate (TMTX) and hydroxyurea (HU)). Such selection can be completed in vitro or in vivo. Transgenic cells and vectors comprising combinations of resistance genes are also provided. Transgenic cells of the embodiments can be used as cell based therapeutics, such as for treatment of HIV infection.

This application claims priority to U.S. Application No. 61/771,331 filed on Mar. 1, 2013, the entire disclosure of which is specifically incorporated herein by reference in its entirety without disclaimer.

### BACKGROUND OF THE INVENTION

1. Field of the Invention

The present invention relates generally to the field of molecular biology and medicine. More particularly, it concerns methods for production and selection of transgenic cells and vectors for the production of such cells.

2. Description of Related Art

Allogeneic transplantation of hematopoietic stem cells (HSCs) has been used successfully to treat a number of hematologic as well as related genetic diseases. This procedure is favored because it can result in the lifelong production of phenotypically normal hematopoietic progeny (Maris 2003 in Beard 2010). This procedure however has an associated high rate of morbidity and mortality caused by graft-versus host disease, complications of immunosuppressive treatments and finding a suitable donor (Otsu 2002 in Beard 2010). A strategy which would circumvent the issues associated with allogeneic HSC transplantation is autologous transplantation of genetically modified cells. However, a limiting factor in such methods is the requirement to obtain a large number of successfully transduced—genetically modified—cells to transplant back to patient. Currently it is difficult to obtain more than 30% of transduced cells. Conversely, a low level of transduction is favorable in the case of gene therapy strategies to prevent multiple insertions of the transgene(s) which could possibly affect oncogenes or tumor suppressors. Thus, there remains a need for methods of producing large numbers of successfully transduced allogeneic cells that could be used in disease therapy, and which provide mechanism(s) for favoring survival of transduced cells following transplantation.

### SUMMARY OF THE INVENTION

In a first embodiment there is provided a method for selecting a transgenic cell comprising (a) obtaining a cell population including cells comprising transgenes for expression of (i) a first resistance gene and (ii) a second resistance gene; and (b) contacting the cell population with an effective amount of a combination of cytotoxic compounds for which the first and second resistance genes will impart resistance upon cells comprising said resistance genes, thereby selecting cells comprising the transgenes. For example, the cell population can be a human cell population, such as a population of human primary cells. In some aspects, the cell population comprises hematopoietic stem cells, such as cells from bone marrow. In further aspects, the cell population comprises stem cells, such as embryonic stem (ES) cells, induced pluripotent stem (iPS) cells or differentiated cells derived from ES or iPS cells. In certain aspects, obtaining a cell population of the embodiments comprises transforming cells with at least a first vector comprising the transgenes or with two or more vectors wherein the first and second resistance genes are comprised on different vectors. In additional aspects, the cell population comprises a further transgene for the expression of a third, fourth or fifth resistance gene. Thus, in some aspects, contacting the cell population with an effective amount of a combination of cytotoxic compounds further comprises contacting the population with cytotoxic compounds for which the third, fourth and/or fifth resistance genes will impart resistance.

A method of selection in accordance with the embodiments can be an in vitro (e.g., wherein cells are selected in culture) or an in vivo method (e.g., wherein the cells are selected in an animal, such as a human subject). Thus, in some aspects, selecting a cell population of the embodiments comprises administering an effective amount of a first and second cytotoxic compound to a subject comprising cells of the embodiments. In further aspects, a method of selecting cells can comprise contacting the cells with the first and second cytotoxic compounds (or administering the first and second cytotoxic compounds) 2, 3, 4, 5, 6, 7, 8, 9, 10 or more times over a period of days, weeks, months or years. In certain aspects, a selection method (in vivo or in vitro) comprises contacting cells (or administering) an amount of drug that is cytotoxic to non-transformed cells when used in conjunction with a further cytotoxic drug, but which, when used alone is not effectively cytotoxic to non-transgenic cells.

In another embodiment there is provided a method for selecting a transgenic cell comprising (a) obtaining a cell population including cells comprising transgenes for expression of (i) a trimetrexate (TMTX) resistance gene and (ii) a hydroxyurea (HU) resistance gene; and (b) contacting the cell population with an effective amount of TMTX and HU, thereby selecting cells comprising the transgenes. For example, the cell population can be a human cell population, such as a population of human primary cells. In some aspects, the cell population comprises hematopoietic stem cells, such as cells from bone marrow. In further aspects, the cell population comprises stem cells, such as embryonic stem cells or induced pluripotent stem (iPS) cells. In certain aspects, obtaining a cell population of the embodiments comprises transforming cells with at least a first vector comprising the transgenes or with two or more vectors wherein the TMTX and HU resistance genes are comprised on different vectors.

A method of selection in accordance with the embodiments can be an in vitro (e.g., wherein cells are selected in culture) or an in vivo method (e.g., wherein the cells are selected in an animal, such as a human subject). Thus, in some aspects, selecting a cell population of the embodiments comprises administering an effective amount of TMTX and HU to a subject comprising cells of the embodiments. In further aspects, a method of selecting cells can comprise contacting the cells with TMTX and HU (or administering TMTX and HU) 2, 3, 4, 5, 6, 7, 8, 9, 10 or more times over a period of days, weeks, months or years. In some aspects, a cell population of the embodiments is subject to selection with at least one cytotoxic drug in vitro and at least one cytotoxic drug in vivo.

In further aspects, a selection method of the embodiments further comprises contacting the cell population with an effective amount of a further cytotoxic drug (e.g., a third drug) wherein the transformed cells in the population do not comprise a transgene for resistance to the further cytotoxic drug. For example, the further drug can be used in an amount effective to kill only non-transgenic cells owing to the non-transgenic cells being sensitized by their susceptibility to the first and/or second cytotoxic compounds. In other words, an effective amount of the third drug can refer to an amount effective to selectively kill non-transgenic cells relative to transgenic cells of the embodiments when applied in the presence of the first and/or second cytotoxic compounds. In yet a further aspect, a selection method of the embodiments comprises administering an effective amount of the third drug and the first and/or the second compound to a subject comprising transgenic cells of the embodiments.

In further aspects, a selection method of the embodiments further comprises contacting the cell population with an effective amount of Busulfan. As used herein an effective amount of a Busulfan refers to an amount effective to selectively kill non-transgenic cells relative to transgenic cells of the embodiments when applied in the presence of TMTX and/or HU. In yet a further aspect, a selection method of the embodiments comprises administering an effective amount of Busulfan and TMTX and/or HU to a subject comprising transgenic cells of the embodiments.

In a further embodiment there is provided a recombinant polynucleotide molecule comprising a RFC1 protein coding sequence and a RRM2 protein coding sequence, wherein both protein coding sequences are operably linked to a promoter. For example, the coding sequences can be operably linked to the same promoter (e.g., and expressed as a polycistronic transcript) or can each be linked to two distinct promoters. Promoter sequences for use according to the embodiments include, without limitation, constitutive promoters, tissue specific promoters and inducible promoters. Further promoters for use according to the embodiments are detailed below. It will further be recognized that a recombinant polynucleotide in accordance with the embodiments may comprise additional sequences that facilitate or regulate expression of encoded transgenes such as, transcription terminators, introns, enhancers and/or polyadenylation signal sequences. In further aspects, a recombinant polynucleotide of the embodiments is comprised in a vector, such as plasmid, episomal or viral vector. For example, the recombinant polynucleotide can be comprised in a retroviral vector such as a lentiviral vector.

Thus, in a further aspect, there is provided a cell comprising a recombinant nucleic acid molecule of the embodiments. For example, the recombinant nucleic acid molecule can be integrated into the genome of the cell or can be maintained in an episomal vector. In certain preferred aspects, a cell of the embodiments comprises no more than 2 copies (or even a single copy) of the recombinant nucleic acid molecule.

Some aspects of the embodiments concern transgenic cell or recombinant polynucleotides that comprise resistance genes, such as TMTX or HU resistance genes. For example, the TMTX resistance gene can be a RFC1 protein coding sequence, such as a sequence encoding a polypeptide at least about 90% identical to human RFC1. Likewise, in some aspects, the HU resistance gene can be a RRM2 protein coding sequence, such as a sequence encoding a polypeptide at least about 90% identical to human RRM2. In certain aspects, the resistance genes (e.g., TMTX and HU resistance genes) are expressed as a polycistronic transcript and are operably linked to a common promoter (e.g., the coding sequence may comprise an internal ribosome entry site (IRES)). In some aspects, the resistance genes (e.g., TMTX and HU resistance genes) are operably linked to two different promoters.

In certain aspects, transgenic cells or recombinant polynucleotides of the embodiments comprise a further transgene. For example, the further transgene can be a transgene that corrects a genetic defect in a cell. In some aspects, the further transgene encodes a polypeptide. In other aspects, the further transgene encodes a functional RNA, such as an RNA that can reduce expression of an endogenous gene (see, e.g., U.S. provisional application No. 61/672,441, incorporated herein by reference). For example, the further transgene can encode an RNA for reducing expression of CCR5. In certain aspects, the further transgene is expressed as a polycistronic transcript with a drug resistance gene (e.g.,

TMTX and/or HU resistance gene). In some aspects, the further transgene is operably linked to a different promoter than the resistance genes (e.g., TMTX and/or HU resistance genes). Additional transgenes for use according to the embodiments include, with limitation, genes encoding adenosine deaminase (ADA), RPE65, beta-globin, lipoprotein lipase (LPL), cystic fibrosis transmembrane conductance regulator (CFTR), tumor suppressors (e.g., P53), growth factors, cell receptors, replication inhibitors, cytokines, transcription factors, hormones, ribozymes, cell transporters, antisense transcripts and inhibitory RNA molecules (e.g., siRNAs or shRNAs).

In yet a further embodiment there is provided a method of treating a subject having a disease comprising administering transgenic cells in accordance to the instant embodiments to the subject. For example, a method of treating a disease is provided comprising (a) obtaining a therapeutic cell population including cells comprising transgenes for expression of (i) a TMTX resistance gene and (ii) a HU resistance gene; and (b) administering the therapeutic cell population to a subject having a disease, wherein the cell population is enriched for cells comprising the transgenes by exposing the cell population to an effective amount of TMTX and HU, thereby selecting cells comprising the transgenes. For example, the cell population can be exposed to TMTX and HU before administration of the cells, after the cells are administered (in vivo) or both before and after the administration. Thus, in some aspects, a method comprises administering TMTX and HU to the subject in an amount effective to selectively enrich cells comprising the transgenes. For example, the TMTX and HU can be administered 2, 3, 4 or more times. In yet a further aspect, a method comprises exposing the cell population to an effective amount of TMTX, HU and Busulfan, such as by administering an effective amount of a mixture of the compounds to the subject.

Thus, in a further embodiment there is provided a method of treating an HIV infection in a subject comprising (a) obtaining a hematopoietic stem cell population from the patient (e.g., a bone marrow cell population); (b) transforming the cell population with one or more vectors comprising transgenes for expression of (i) a Trimetrexate (TMTX) resistance gene; (ii) a Hydroxyurea (HU) resistance gene; and (iii) a transgene that encodes an RNA for reducing expression of CCR5; and (c) administering the transformed cell population to the subject, wherein the cell population is enriched for cells comprising the transgenes by exposing the cell population to an effective amount of TMTX and HU, thereby selecting cells comprising the transgenes. In a further aspect, the cell population is enriched for the cells comprising the transgenes by exposing the cell population to an effective amount of TMTX, HU, and Busulfan. In some aspects, the method further comprises exposing the subject to radiation to ablate bone marrow cells after obtaining the bone marrow cell population from the subject.

In yet a further embodiment a composition is provided for use in the treatment of a disease comprising a recombinant nucleic acid molecule or a cell population of the embodiments. For example, the recombinant nucleic acid or cell population can comprise transgenes for expression of (i) a TMTX resistance gene; (ii) a HU resistance gene; and (iii) therapeutic gene.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Synergistic killing effect of Hydroxyurea (HU) and Trimetrexate (TMTX) on Hela cells. Hela cells were either treated with 100 μM HU (100 uM HU), 1 nM TMTX or a combination of both compounds. Control cells (Control) no compound was added. After 5 days cells were harvested and stained with Calcein and cell survival was determined Percentages of survival cells were normalized to control cells.

FIG. 2: Selective enrichment of Hela cells expressing HU and TMTX resistance gene as compared to control Hela cells expressing GFP. Control and transduced Hela cells were incubated with HU and TMTX for 3 days, after which survival rate of both cell population were measured by FACS analysis.

FIG. 3: In vivo selective enrichment of blood cells expressing HU and TMTX resistance gene. Bone marrow transplantation experiments were performed in C57/b16 mice. Total bone marrow was obtained from large bones of C57/B16 mice and maintained for 24 h in IMDM medium supplemented with 100 ng/ml of SCF. Bone marrow cells were then transduced with 10⁹ lentivector particles for 8 hours before being injected into the tail vein of irradiated C57/B16 mice. Six to eight weeks later, blood samples were taken and GFP expression was measured by FACS. After which mice were injected intraperitoneally with either HU or TMTX with initial doses of HU of 200, 500 and 1000 mg/kg per day and initial doses of TMTX of 10, 25, 50 mg/kg with 2 injections per week. GFP expressing blood cells were measured once a week. (A) Irradiated mice, transplanted with bone marrow cells and transduced with a lentivector expressing GFP and the hydroxyurea resistance (GFP-HU). A transient increase in GFP-positive leukocytes (up to 8%) was observed in hydroxyurea-treated mice (individual lines depict individual mice).

### DESCRIPTION OF ILLUSTRATIVE EMBODIMENTS

### I. The Present Invention

For many years now gene therapy has been examined as a possible method for treating a wide range of diseases. However, technical impediments have thus far prevented most therapies from reaching their full potential. One area of intense research involves cell-based therapies that employ cells harvested from a patient and that are engineered ex vivo prior to re-implantation. Such methods have the potential advantage of avoiding immune rejection (which can occur when non-self cells are employed). However, the process of successfully and safely “engineering” cells is technically challenging. Likewise, in most cases, a large number of cells are required in order for the re-implanted cells to have any effect on a given disease. Thus, there remains a need for new cell engineering methods and vector to improve gene and cell-based therapies.

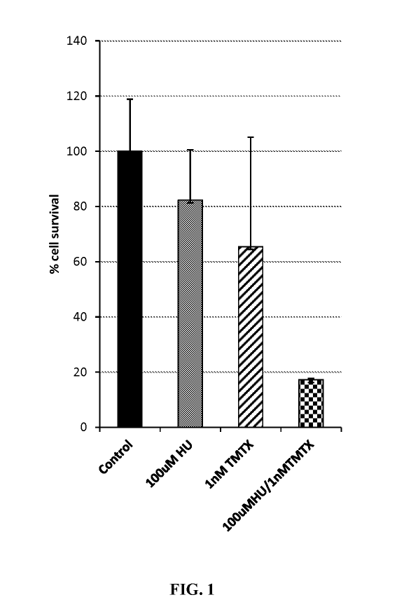

The studies detailed here address two of the most crucial impediments to engineered cell-based therapies by providing effective methods for selection of engineered cells. As demonstrated here, transgenic cells that comprise genes for resistant to TMTX and HU can be efficiently enriched using low concentrations of the cytotoxic compounds. Importantly, resistance to TMTX and HU is conferred by endogenous human genes (RFC1 and RRM2) thus transgenic cells will not produce a cell-mediated immune response as could occur when using non-human drug resistance genes. While these transgenic cells survive and continue to proliferate, non-transgenic cells are killed by the combined cytotoxic effect of the compounds (see, e.g., FIG. 1). This efficient selection allows a relatively small number of transgenic cells to be expanded while selectively removing non-transgenic cells. Furthermore, the use of two or more selection drugs allows the each drug to be used at a concentration or dosage that would not normally be effectively cytotoxic, which may eliminate or minimize side effects from in vivo selection. Crucially, TMTX and HU are approved for use in humans and thereby amenable for in vivo selection of cells. Accordingly, even if only a small number of cells are available for implantation, in vivo selection can allow for selective expansion of a transgenic cell population in the patient. As an additional safety feature the RFC1 gene confers Methotrexate sensitivity to expressing cells to thereby allows transgenic cells to be easily and selectively killed in a patient (if a problem with the cells arises). Thus, unique combination selection techniques provided can be used to develop powerful new cell-based therapies for disease treatment.

New vector and cell selection systems provided here could be used to produce a vast array of different types of transgenic cells. As an example, the techniques detailed here can be used in a therapy for HIV infection. To date only one patient (the “Berlin patient”) has ever been know to be successfully cured of HIV infection. The successful therapy was coincidentally applied as part of a treatment for leukemia. Specifically, the patient was given an HLA-matched blood stem cell transplant after receiving whole body irradiation to kill-off all endogenous bone marrow cells. The donor cells in the case were from a subject comprising homozygous deletions in the CCR5 gene delta 32/delta 32 (see e.g., Hutter et al., 2009), a genetic trait known to render individuals highly resistant to HIV infection. This patient recovered from the transplant (and a subsequent transplant upon recurrence of the leukemia) and was still demonstrated HIV free without antiretroviral therapy more than 5 years after the initial transplant. The results achieved by the therapy were nothing short of amazing. Unfortunately, however, the therapy is not amenable to use on a mass scale given the difficulty in identifying HLA-matched donors for blood cell transplantation, to say nothing of finding such a matching donor that also has a homozygous CCR5 disruption (which only appears to be present in some 1% of Northern Europeans).

Methods of the embodiments, however, provide an alternative to this proven and successful therapy. In this case blood stem cells (from bone marrow) are harvested from the HIV patient. The cells are then transduced ex vivo with a lentiviral vector encoding an expressible RFC1 and RRM2 gene along with an expressible RNA that down regulates CCR5 expression. Upon transduction, the cells are selected with TMTX and HU to enrich the cell population for transgenic cells. The cell population is then reintroduced into the patient (either with or without first irradiating the patient to ablate blood stem cells). After reintroduction the transgenic cells can be further expanded relative to non-transgenic cells by administering TMTX and HU to the patient. In this way any residual HIV virus that remains in the patient is unable to infect the transgenic cells with impaired CCR5 expression. Thus, the new methods provided here enable a previously successful HIV therapy to be adapted such that it can be used on a much broader scale, for the first time accessible to a wide population of HIV-positive patients.

The techniques detailed here are likewise applicable to other cell-based therapies. For example, the techniques can be applied to cell-based gene therapies wherein a genetic defect is corrected in cells of a patient (e.g., primary cells, patient derived iPS cells or differentiated iPS cells) and the modified cells are reimplanted into the patient. Selection methods detailed herein allow for the selective expansion of the genetically modified cells for a far more effective reimplantation that does not require the ex vivo growth of massive amounts of cell material. In the case of a genetic liver defect, for example, cells can be harvested from a patient, transduced to correct the genetic defect and introduce drug resistance genes. Transduced cells are then selected (e.g., both in vitro and in vivo) thereby allowing selective expansion of what may be a small initial cell population in the patient. Accordingly, the percent of cells in the patient's liver with the corrected defect can be steadily increased thereby providing therapeutic effect.

### II. Cytotoxic Agents and Cell Selection Systems

As detailed above, some aspects of the embodiments concern transgenic cells and expression vectors (e.g., lentiviral vectors) that comprise a drug resistance gene. Some preferred resistance genes and drugs for selection of the same are detailed below.

### Hydroxyurea

HU is a chemotherapeutic agent and has a potent effect in the bone marrow; it has been used to treat human haematologic malignancies, sickle cell anaemia and has also been described as having antiretroviral properties (Ravot et al., 1999; Charache et al., 1995 in Ravot; Lori et al., 2007). Hydroxyurea acts by inhibiting the ribonucleotide reductase M2 subunit thereby halting synthesis of dNTPS. The human ribonucleotide reductase enzyme is responsible for production of dNTPs and the enzyme consists of two subunits, M1 & M2, which are inactive when alone. The M1 protein is expressed from the RRM1 gene and levels of M1 protein are constant in all phases of the cell cycle. M2 protein comes from the RRM2 gene and the M2 protein is the limiting factor and thus controls ribonucleotide reductase activity. It has been described that cells which over express M2 protein have a resistance to the drug Hydroxyurea (Ravot, 1999; Yang Feng, 1987). The sequence for RRM2 is well known and is provided, for example, as NCBI accession nos. NP_{—}001159403 and NP_{—}001025, each incorporated herein by reference.

### Trimetrexate

TMTX and methotrexate (MTX) are antifolate drugs which inhibit the dihydrofolate reductase enzyme (DHFR). TMTX has been used to treat pneumocystis pneumonia in HIV patients and MTX has been used extensively to treat acute lymphocytic leukemia, non-Hodgkin's lymphoma, osteosarcoma, choriocarcinoma, breast cancer, and head and neck cancer (Allegra, 1987; Schornagel, 1983 in Fry, 1988). Trimetrexate has also been shown to effectively kill myelotoxic resistant tumor cells (Lacerda, 1995 in Spencer, 1996). Inhibition of DHFR by antifolate drugs results in cells not being able to reduce folic acids and thus DNA synthesis, cell proliferation, and growth is stopped. Human cells also have folate transport mechanisms and one such transporter is the reduced folate carrier (RFC), which is transcribed from the human RFC gene. RFCs are transmembrane glycoproteins and carry reduced folates into cells, however, they also are able to transport antifolate drugs such as MTX into cells thereby increasing cell sensitivity to MTX (Liu, 2002). TMTX on the other hand is a lipid soluble compound and diffuses into cells independently of the RFC system (Fry, 1988). Therefore it has been described that overexpression of the RFC gene causes cells to become resistant to TMTX as they can rescue themselves by increased transport of exogenous folates into the cell (Liu, 2002). The sequence encoding RFC is well known and is provided, for example, as NCBI accession no. NM_{—}194255.2, incorporated herein by reference.

### Busulfan

Busulfan (marketed as Myleran®, GlaxoSmithKline or IV Busulfex®, Otsuka America Pharmaceuticals Inc.) is a cancer drug that has been used as a mainstay for treatment of chronic myeloid leukemia (CML). Busulfan is a cell cycle non-specific alkylating antineoplastic agent, in the class of alkyl sulfonates. Its chemical designation is 1,4-butanediol dimethanesulfonate. It is contemplated that Busulfan may be used in conjunction with TMTX and HU for selection of transgenic cells in accordance with the embodiments. While TMTX and HU resistance genes do not provide resistance to Busulfan per se, it is contemplated that a concentration of the drug can be used that would alone not have effective toxicity on cell populations. However, the additional use of the TMTX and/or HU selection reagents will selectively sensitize non-transgenic cells to Busulfan toxicity. Accordingly, as used herein an effective amount of Busulfan refers to an amount effective to selectively kill non-transgenic cells when applied in the presence of TMTX, HU, or a combination thereof

### Additional Selection Drugs and Markers

Additional selectable genes that can be used according to embodiments include those that confer resistance to neomycin, puromycin, blastocidin, G418, hygromycin B, mycophenolic acid, bleomycin or histidinol. Thus, in certain aspects, cells or vectors of the embodiments comprise a nucleic acid molecule for the expression of a puromycin N-acetyl-transferase gene, a blasticidin S deaminase (bsd), an aminoglycoside phosphotransferase (e.g., neo^{r}), a hygromycin resistance gene (e.g., hyg or hph), a xanthine guanine phosphoribosyltransferase (XGPRT), Ba Sh ble gene or a histidinol dehydrogenase (hisD).

Additional cytotoxic compounds that are contemplated for use with the present embodiments include, without limitation, Abacavir, Lamuvudine, Zidovudine, Aldesleukin, Altretamine, Amphotericin B (systemic), Amphotericin B cholesteryl complex, Amphotericin B lipid complex, Amphotericin B liposomal complex, Anastrazole, Azathioprine, Bexarotene, Busulfan, Capecitabine, Carboplatin, Carmustine (systemic), Chlorambucil, Chloramphenicol, Clozapine, Cisplatin, Cladribine, Colchicine, Cyclophosphamide, Cytarabine, Dacarbazine, Dactinomycin, Daunorubicin, Daunorubicin (liposomal), Didanosine, Docetaxel, Doxorubicin, Doxorubicin (liposomal), Eflornithine, Epirubicin, Etoposide, Floxuridine, Flucytosine, Fludarabine, Fluorouracil (systemic), Ganciclovir, Gemcitabine, Gemtuzumab Ozogamicin, Hydroxyurea, Idarubicin, Ifosfamide, Imatinib, Interferon (gamma), Interferons (alpha), Irinotecan, Lomustine, Mechlorethamine (systemic), Melphalan, Mercaptopurine, Methotrexate, Mitomycin, Mitoxantrone, Paclitaxel, Pegaspargase, Pentostatin, Plicamycin, Procarbazine, Sodium iodide I 131, Sodium phosphate P 32, Strontium 89 chloride, Streptozocin, Temozolomide, Teniposide, Thioguanine, Thiotepa, Topotecan, Trimetrexate, Uracil mustard, Valganciclovir, Valrubicin, Vidarabine (systemic, with high doses), Vinblastine, Vincristine, Vinorelbine, Zidovudine, Zidovudine and Lamivudine, and Zoledronic Acid. Transgenes providing resistance to any of the foregoing drugs are likewise contemplated for use according to the embodiments.

### III. Vectors for Cloning, Gene Transfer and Expression

Within certain aspects, expression vectors are employed to express a nucleic acid of interest, such as a nucleic acid that inhibits the expression of a particular gene. Expression requires that appropriate signals be provided in the vectors, and which include various regulatory elements, such as enhancers/promoters from both viral and mammalian sources that drive expression of the genes of interest in host cells. Elements designed to optimize RNA stability in host cells also are defined. The conditions for the use of a number of dominant drug selection markers for establishing permanent, stable cell clones expressing the products are also provided, as is an element that links expression of the drug selection markers to expression of the polypeptide.

### A. Regulatory Elements

Throughout this application, the term “expression construct” or “expression vector” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest, i.e., as is the case with inhibitory RNA molecules of the embodiments.

Eukaryotic expression cassettes included in the nucleic acid molecules of the embodiments preferably contain (in a 5′-to-3′ direction) an eukaryotic transcriptional promoter operably linked to a protein-coding sequence, splice signals including intervening sequences, and a transcriptional termination/polyadenylation sequence.

### 1. Promoter/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term promoter will be used here to refer to a group of transcriptional control modules that are clustered around the initiation site for eukaryotic RNA polymerase (Pol) I, II or III. Much of the thinking about how promoters are organized derives from analyses of several viral Pol II promoters, including those for the HSV thymidine kinase (tk) and SV40 early transcription units. These studies, augmented by more recent work, have shown that promoters are composed of discrete functional modules, each consisting of approximately 7-20 by of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 by apart before activity begins to decline. Depending on the promoter, it appears that individual elements can function either co-operatively or independently to activate transcription.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, in order to regulate the expression of the gene of interest. This list is not intended to be exhaustive of all the possible elements involved in the promotion of gene expression but, merely, to be exemplary thereof In some aspects, a promoter for use according to the instant embodiments is a non-tissue specific promoter, such as a constitutive promoter.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

A wide range of viral promoters, cellular promoters/enhancers and inducible promoters/enhancers may be used in constructs of the embodiments. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct. In some aspects a promoter for use according to the embodiments is a promoter that is active in human hematopoietic stem cells (hHSCs) or their in early progenitors. In further aspects, a promoter may a be a promoter that is active in primarily in differentiated cells or cells of a particular lineage or tissue.

Examples of promoters for use according to the embodiments include, with limitation, the promoters for EF1, Ubiquitin, hPGK, Oct3/4, Sox2, Nanog, GATA2, Runx1, SCL/TAL-1, LMO-2, Mixed lineage leukemia (MLL), TEL/ETV6, GFII, CD34, Phosphoglycerate kinase, Wiskott-Aldrich syndrome (WAS), Immunoglobulin Heavy Chain, Immunoglobulin Light Chain, T Cell Receptor, HLA DQa, HLA DQβ, β-Interferon, Interleukin-2, Interleukin-2 Receptor, MHC Class II 5, MHC Class II HLA-DRa, β-Actin, Muscle Creatine Kinase (MCK), Prealbumin (Transthyretin), Elastase I, Metallothionein (MTII), Collagenase, Albumin, α-Fetoprotein, t-Globin, β-Globin, c-fos, c-HA-ras, Insulin, Neural Cell Adhesion Molecule (NCAM), α1-Antitrypain, H2B (TH2B) Histone, Type I Collagen, Glucose-Regulated Proteins (GRP94 and GRP78), Human Serum Amyloid A (SAA), Troponin I (TN I), Platelet-Derived Growth Factor (PDGF) and Duchenne Muscular Dystrophy. In some aspects, a viral promoter, such as a promoter from SV40, Polyoma virus, Retroviruses (e.g., RSV), Papilloma Virus, Hepatitis B Virus, Human Immunodeficiency Virus or Cytomegalovirus (CMV) can be used.

### 2. Initiation Signals and Internal Ribosome Binding Sites

In the case of a sequence coding for a protein, a specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### 3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999; Levenson et al., 1998; and Cocea, 1997, incorporated herein by reference). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### 4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference).

### 5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that the terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### 6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

### 7. Origins of Replication

In order to propagate a vector in a host cell, it may contain one or more origins of replication sites (often termed “ori”), for example, a nucleic acid sequence corresponding to oriP of EBV as described above, which is a specific nucleic acid sequence at which replication is initiated. Alternatively a replication origin of other extra-chromosomally replicating virus as described above or an autonomously replicating sequence (ARS) can be employed.

### 8. Selection and Screenable Markers

As detailed above, in certain aspects one or more selectable marker(s) is incorporated into a vector or cell of the embodiments. In certain aspects of the embodiments, cells containing a nucleic acid construct of the present invention may be identified or selected in vitro or in vivo by including a marker in the expression cassette. Such markers would confer an identifiable change to the cell permitting easy identification of cells containing the expression cassette. Generally, a selection marker is one that confers a property that allows for selection.

Additional selectable genes that can be used according to embodiments include those that confer resistance to neomycin, puromycin, blastocidin, G418, hygromycin

B, mycophenolic acid, bleomycin or histidinol. Thus, in certain aspects, cells or vectors of the embodiments comprise a nucleic acid molecules for the expression of a puromycin N-acetyl-transferase gene, a blasticidin S deaminase (bsd), an aminoglycoside phosphotransferase (e.g., neo^{r}), a hygromycin resistance gene (e.g., hyg or hph), a xanthine guanine phosphoribosyltransferase (XGPRT), Ba Sh ble gene or a histidinol dehydrogenase (hisD).

In addition to markers conferring a phenotype that allows for the discrimination of transformants based on the implementation of conditions, other types of markers including screenable markers such as GFP, whose basis is colorimetric analysis, are also contemplated. For example, cells expressing a fluoresence marker, such as GFP can be rapidly separated from a mixed cell population by fluorescence activated cell sorting (FACS). Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Certain embodiments of the present invention utilize screenable reporter genes to indicate specific property of cells, for example, differentiation along a defined cell lineage by activating a condition-responsive regulatory element which controls the reporter marker gene expression.

Examples of such reporters include genes encoding cell surface proteins (e.g., CD4, HA epitope), fluorescent proteins, antigenic determinants and enzymes (e.g., β-galactosidase or a nitroreductase). The vector containing cells may be isolated, e.g., by FACS using fluorescently-tagged antibodies to the cell surface protein or substrates that can be converted to fluorescent products by a vector encoded enzyme. In certain aspects cell-permeable dyes can be used to identify cells expressing a reporter. For example, expression of a NFAT nitroreductase gene can be detected by using a cell permeable pro-fluorogenic substrate such as CytoCy5S (see, e.g., U.S. Pat. Nos. 5,633,158, 5,780,585, 5,977,065 and EP Patent No. EP 1252520, each incorporated herein by reference).

### IV. Delivery of Nucleic Acid Molecules and Expression Vectors

In certain aspects, vectors for delivery of nucleic acids of the embodiments could be constructed to express these factors in cells. In a particular aspect, the following systems and methods may be used in delivery of nucleic acids to desired cell types.

### A. Homologous Recombination

In certain aspects of the embodiments, the vectors encoding nucleic acid molecules of the embodiments may be introduced into cells in a specific manner, for example, via homologous recombination. Current approaches to express genes in stem cells have involved the use of viral vectors (e.g., lentiviral vectors) or transgenes that integrate randomly in the genome. These approaches have not been successful due in part because the randomly integrated vectors can activate or suppress endogenous gene expression, and/or the silencing of transgene expression. The problems associated with random integration could be partially overcome by homologous recombination to a specific locus in the target genome.

Homologous recombination (HR), also known as general recombination, is a type of genetic recombination used in all forms of life in which nucleotide sequences are exchanged between two similar or identical strands of DNA. The technique has been the standard method for genome engineering in mammalian cells since the mid 1980s. The process involves several steps of physical breaking and the eventual rejoining of DNA. This process is most widely used in nature to repair potentially lethal double-strand breaks in DNA. In addition, homologous recombination produces new combinations of DNA sequences during meiosis, the process by which eukaryotes make germ cells like spermatozoa and ova. These new combinations of DNA represent genetic variation in offspring which allow populations to evolutionarily adapt to changing environmental conditions over time. Homologous recombination is also used in horizontal gene transfer to exchange genetic material between different strains and species of bacteria and viruses. Homologous recombination is also used as a technique in molecular biology for introducing genetic changes into target organisms.

Homologous recombination can be used as targeted genome modification. The efficiency of standard HR in mammalian cells is only 10⁻⁶ to 10⁻⁹ of cells treated (Capecchi, 1990). The use of meganucleases, or homing endonucleases, such as I-SceI have been used to increase the efficiency of HR. Both natural meganucleases as well as engineered meganucleases with modified targeting specificities have been utilized to increase HR efficiency (Pingoud and Silva, 2007; Chevalier et al., 2002). Another path toward increasing the efficiency of HR has been to engineer chimeric endonucleases with programmable DNA specificity domains (Silva et al., 2011). Zinc-finger nucleases (ZFN) are one example of such a chimeric molecule in which Zinc-finger DNA binding domains are fused with the catalytic domain of a Type IIS restriction endonuclease such as Fold (as reviewed in Durai et al., 2005; PCT/US2004/030606). Another class of such specificity molecules includes Transcription Activator Like Effector (TALE) DNA binding domains fused to the catalytic domain of a Type IIS restriction endonuclease such as FokI (Miller et al., 2011: PCT/IB2010/000154).

### B. Nucleic Acid Delivery Systems

One of skill in the art would be well equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc.), lentiviral vectors (e.g., derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

### 1. Episomal Vectors

The use of plasmid- or liposome-based extra-chromosomal (i.e., episomal) vectors may be also provided in certain aspects of the invention, for example, for reprogramming of somatic cells. Such episomal vectors may include, e.g., oriP-based vectors, and/or vectors encoding a derivative of EBV-protein EBNA-1. These vectors may permit large fragments of DNA to be introduced to a cell and maintained extra-chromosomally, replicated once per cell cycle, partitioned to daughter cells efficiently, and elicit substantially no immune response.

In particular, EBNA-1, the only viral protein required for the replication of the oriP-based expression vector, does not elicit a cellular immune response because it has developed an efficient mechanism to bypass the processing required for presentation of its antigens on MHC class I molecules (Levitskaya et al., 1997). Further, EBNA-1 can act in trans to enhance expression of the cloned gene, inducing expression of a cloned gene up to 100-fold in some cell lines (Langle-Rouault et al., 1998; Evans et al., 1997). Finally, the manufacture of such oriP-based expression vectors is inexpensive.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV); Herpes virus saimiri (HS) and Marek's disease virus (MDV). Also other sources of episome-based vectors are contemplated, such as yeast ARS, adenovirus, SV40, or BPV.

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

### 2. Transposon-Based System

According to a particular embodiment the introduction of nucleic acids may use a transposon—transposase system. The used transposon—transposase system could be the well known Sleeping Beauty, the Frog Prince transposon—transposase system (for the description of the latter see e.g., EP1507865), or the TTAA-specific transposon piggyback system.

Transposons are sequences of DNA that can move around to different positions within the genome of a single cell, a process called transposition. In the process, they can cause mutations and change the amount of DNA in the genome. Transposons were also once called jumping genes, and are examples of mobile genetic elements.

There are a variety of mobile genetic elements, and they can be grouped based on their mechanism of transposition. Class I mobile genetic elements, or retrotransposons, copy themselves by first being transcribed to RNA, then reverse transcribed back to DNA by reverse transcriptase, and then being inserted at another position in the genome. Class II mobile genetic elements move directly from one position to another using a transposase to “cut and paste” them within the genome.

### 3. Viral Vectors

In generating recombinant viral vectors, non-essential genes are typically replaced with a gene or coding sequence for a heterologous (or non-native) protein or nucleic acid. Viral vectors are a kind of expression construct that utilizes viral sequences to introduce nucleic acid and possibly proteins into a cell. The ability of certain viruses to infect cells or enter cells via pH-dependent or pH-independent mechanisms, to integrate their genetic cargo into a host cell genome and to express viral genes stably and efficiently have made them attractive candidates for the transfer of foreign nucleic acids into cells (e.g., mammalian cells). Non-limiting examples of viral vectors that may be used to deliver a nucleic acid of certain aspects of the present invention are described below.

Retroviruses have promise as gene delivery vectors due to their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid (i.e., the vector genome) to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Depending on the tropism of the envelope protein used to cover the vector particles surface, retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et cd., 1997; Giry-Laterriere et al., 2011; U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and that is described in U.S. Pat. No. 5,994,136, incorporated herein by reference.

### C. Nucleic Acid Delivery

Introduction of a nucleic acid, such as DNA or RNA, into cells to be programmed with the current invention may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

### 1. Liposome-Mediated Transfection

In a certain embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen). The amount of liposomes used may vary upon the nature of the liposome as well as the cell used, for example, about 5 to about 20 ug vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

### 2. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

### 3. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

### 4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

D. Cell culturing

Generally, cells of the present invention are cultured in a culture medium, which is a nutrient-rich buffered solution capable of sustaining cell growth.

Culture media suitable for isolating, expanding and differentiating stem cells according to the method described herein include but not limited to high glucose Dulbecco's Modified Eagle's Medium (DMEM), DMEM/F-12, Liebovitz L-15, RPMI 1640, Iscove's modified Dubelcco's media (IMDM), and Opti-MEM SFM (Invitrogen Inc.). Chemically Defined Medium comprises a minimum essential medium such as Iscove's Modified Dulbecco's Medium (IMDM) (Gibco), supplemented with human serum albumin, human Ex Cyte lipoprotein, transferrin, insulin, vitamins, essential and non-essential amino acids, sodium pyruvate, glutamine and a mitogen is also suitable. As used herein, a mitogen refers to an agent that stimulates cell division of a cell. An agent can be a chemical, usually some form of a protein that encourages a cell to commence cell division, triggering mitosis. In one embodiment, serum free media such as those described in U.S. Ser. No. 08/464,599 and WO96/39487, and the “complete media” as described in U.S. Pat. No. 5,486,359 are contemplated for use with the method described herein. In some embodiments, the culture medium is supplemented with 10% Fetal Bovine Serum (FBS), human autologous serum, human AB serum or platelet rich plasma supplemented with heparin (2 U/ml). Cell cultures may be maintained in a CO₂ atmosphere, e.g., 5% to 12%, to maintain pH of the culture fluid, incubated at 37° C. in a humid atmosphere and passaged to maintain a confluence below 85%.

### V. Pharmaceutical Compositions and Routes of Administration

Where clinical application of cells or cytotoxic therapies (e.g., in vivo selection) is undertaken, it will be necessary to prepare compositions as a pharmaceutical formulations appropriate for the intended application. Generally, this will entail preparing a pharmaceutical composition that is essentially free of pyrogens, as well as any other impurities that could be harmful to humans or animals. One also will generally desire to employ appropriate buffers to render the complex stable and allow for uptake by target cells. Aqueous compositions of the embodiments, for instance, can comprise effective amount of a cytotoxic compound of a combination thereof as discussed above, further dispersed in pharmaceutically acceptable carrier or aqueous medium. Such compositions also are referred to as inocula. The phrases “pharmaceutically” or “pharmacologically acceptable” refer to compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms. The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well-known parameters. Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions generally will take the form of solutions or suspensions.

The therapeutic compositions of the present embodiments may include classic pharmaceutical preparations. Administration of therapeutic compositions according to the present invention will be via any common route so long as the target tissue is available via that route. In this case, intravenous injection or infusion may be preferred. Such compositions would normally be administered as pharmaceutically acceptable compositions that include physiologically acceptable carriers, buffers or other excipients.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., in the case of cytotoxic compositions the appropriate route and dose to achieve selective killing of non-transduced cells.

In some aspects, an effective dose range of a therapeutic can be extrapolated from effective doses determined in animal studies. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ, factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25.Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Combined Hydroxyurea and Trimetrexate

### Killing Effect of Hydroxyurea and Trimetrexate on Hela Cells

Hela cells were cultured in DMEM medium. To assess the killing effect of Hydroxyurea (HU) and Trimetrexate (TMTX), cells were incubated with either HU or TMTX or both at final concentrations of 100 μM HU and 1 nM TMTX for 5 days. Cells were then washed with PBS, harvested and stained with Calcein (Invitrogen) according to the manufacturer specifications to determine cell survival.

### Construction of a New Generation Mir-16 Lentivectors

PCR reactions conditions were set up according to the manufacturer specifications (Agilent Technologies, Santa Clara, USA) and using The Herculase II Fusion DNA Polymerase. PCR products were ligated, using T4 DNA ligase (New England Biolabs, Ipswich, Mass.), into a pENTR Gateway entry plasmid containing the Green fluorescent protein (GFP) coding sequence (pENTR-GFP). The final lentivector expressing-GFP and the Hydroxyurea or Trimetrexate resistance genes was constructed by carrying out a Gateway LR reaction with HIV-1 derived 2nd generation vector backbone, pCLX.

### Virus Production and Titration

Lentiviral vector stocks were generated by transient transfection of

HEK 293T cells (Salmon & Trono, 2007) with the HIV-1 derived packaging psPAX2 and the envelope pCAG-VSVG plasmids. Lentiviral titer was assessed by flow cytometry. Hela cells were transduced with a GPF report gene and GPF fluorescence was measured 5 days after transduction.

### Hydroxyurea and Trimetrexate Cytotoxic Assay

Hela cells were transduced with lentivector expressing GFP and the Hydroxyurea or Trimetrexate resistance genes. Three to five days after transduction, cell transduction efficiency was determined by measuring GFP fluorescence. Transduced Hela cells were treated with the appropriate concentration of cytotoxic compounds and survival rate were measured by FACS analysis.

### Results

Hela cells were treated as outlined above with 100 μM HU and 1 nM TMTX or a combination of both compounds (for control cells no compound was added). After 5 days cells were harvested, stained with Calcein and cell survival was determined and plotted. As shown in FIG. 1 HU and TMTX, when combined and even at low concentration were able to synergize in efficient killing of Hela cells. These studies indicate that the combination of these two compounds in particular will be highly efficient for cell selection and by virtue of the low concentrations that can be used may be uniquely adapted for in vivo selection.

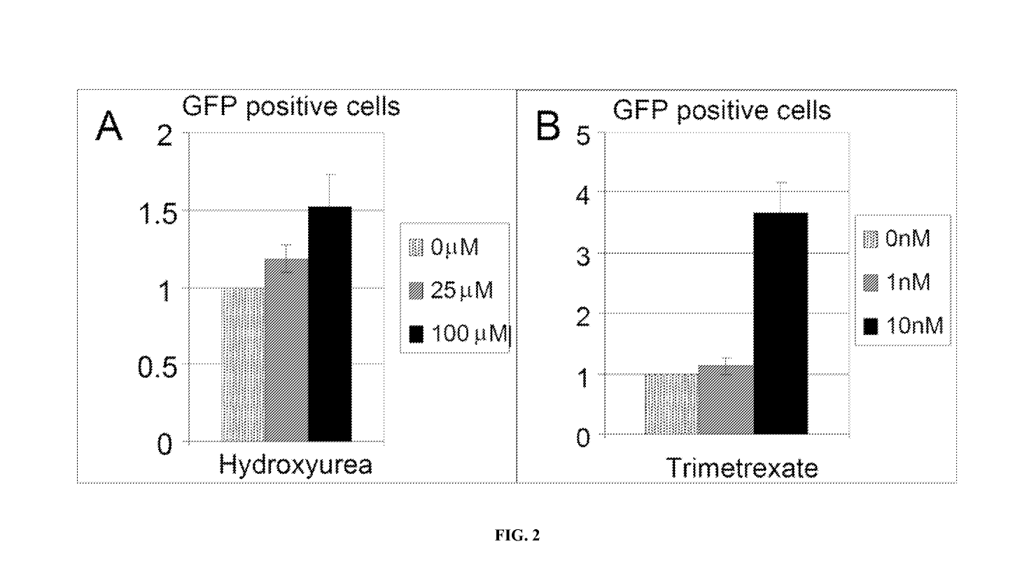

Next, the ability of either HU or TMTX to selectively enrich transduced Hela cells was evaluated by FACS. Hela cells were transduced as outlined above and then cultured in control media or media including 25 μM HU, 100 μM HU, 1 nM TMTX or 10 nM TMTX. After culture in the indicated conditions for three days, the proportion of transduced cells (GFP positive) versus non-transduced cells was determined by FACS and the results plotted. Results shown in FIG. 2 demonstrate modest selection could be achieved with HU at both tested concentrations. TMTX on the other hand only resulted in significant selection when applied at 10 nM level.

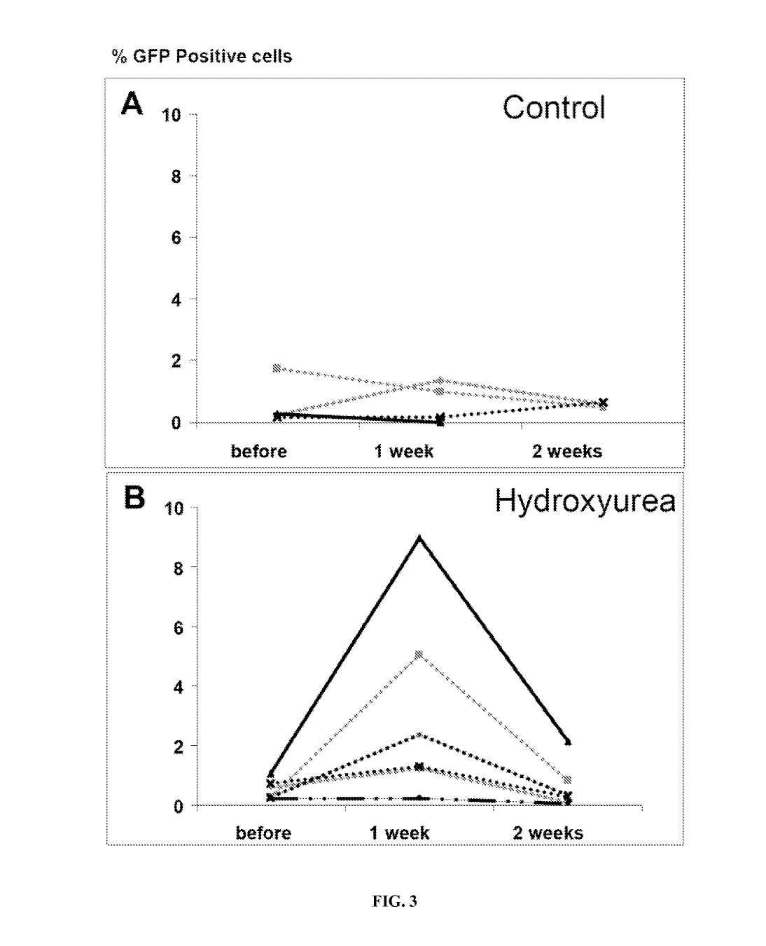

In order to more directly assess the ability of HU and TMTX to select cells in vivo studies were undertaken in mice injected with transduced cells. Briefly, for transduction total bone marrow was obtained from large bones of C57/B16 mice and maintained for 24 h in IMDM medium supplemented with 100 ng/ml of SCF. The bone marrow cells were then transduced with lentivector particles (1×10⁹) for 8 hours before being injected into the tail vein of irradiated C57/B16 mice. Six to eight weeks later, blood samples were taken and GPF expression was measured by FACS. After which mice were injected intraperitoneally with either HU or TMTX with initial doses of HU of 200, 500, and 1000 mg/kg per day and initial doses of TMTX of 10, 25, 50 mg/kg with 2 injections per week. GFP expressing blood cells were measured once a week. Initial results of these studies are shown in FIG. 3 and demonstrate that treatment of the mice with HU resulted in a transient increase of circulating GFP positive cells, such that in some treated animals 5%-10% of the cells were GFP positive one week after HU injection.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Fleenor et al., Comparison of human and Xenopus GATA-2 promoters, Gene, 179:219-223, 1996.

Pozner et al., Developmentally regulated promoter-switch transcriptionally controls Runxl function during embryonic hematopoiesis, BMC Dev. Biol., 7:84, 2007.

Aplan et al., The SCL gene is formed from a transcriptionally complex locus, Mol. Cell. Biol., 10:6426-6435, 1990.

Courtes et al., Erythroid-specific inhibition of the ta1-1 intragenic promoter is due to binding of a repressor to a novel silencer, J. Biol. Chem., 275:949-958, 2000.

Pruess et al., Promoter 1 of LMO2, a master gene for hematopoiesis, is regulated by the erythroid specific transcription factor GATA1, Gene Function and Disease, 1:87-94, 2000.

Horiguchi et al., Transcriptional Activation of the Mixed Lineage Leukemia—p27Kip1 Pathway by a Somatostatin Analogue, Clin. Cancer Res., 15:2620, 2009.

Montpetit and Sinnett, Comparative analysis of the ETV6 gene in vertebrate genomes from pufferfish to human, Oncogene, 3437-3442, 2001.

Liu and Cowell, Cloning and characterization of the TATA-less promoter from the human GFI1 proto-oncogene, Ann. Hum. Genet., 64:83-86, 2000.

Burn et al., The human CD34 hematopoietic stem cell antigen promoter and a 3′ enhancer direct hematopoietic expression in tissue culture, Blood, 80:3051-3059, 1992.

Leuci et al., Stem Cells, 27:2815-2823, 2009.

Wiborg et al., EMBO J., 4:755-759, 1985.

Varma et al., J. Stem Cell Regen. Med., 7:41-53, 2011.

Tokushige et al., J. Virol. Methods, 64:73-80, 1997.

Qin et al., PLoS One, 5:e10611, 2010.

Hong et al., Mol. Ther., 15:1630-1639, 2007.

1. An in vitro method for selecting a transgenic cell comprising:
(a) obtaining a cell population including cells comprising transgenes for expression of (i) a first resistance gene and (ii) a second resistance gene; and
(b) contacting the cell population with an effective amount of the toxic compounds to which the first and second resistance genes render the transgenic cells resistant, thereby selecting cells comprising the transgenes.

2. The method of claim 1, wherein the cell population is a human cell population.

3. The method of claim 2, wherein the cell population comprises primary cells.

4. The method of claim 3, wherein the cell population comprises cells extracted from bone marrow.

5. The method of claim 1, wherein the cell population comprises stem cells or induced pluripotent cells (iPS).

6. The method of claim 1, wherein obtaining the cell population comprises transforming cells with at least a first vector comprising the transgenes.

7. The method of claim 6, wherein the vector is a plasmid vector, an episomal vector, or a viral vector.

8. The method of claim 7, wherein the viral vector is a lentiviral vector.

9. The method of claim 1, wherein the cells comprise a further transgene.

10. The method of claim 9, wherein the further transgene corrects a genetic defect in the cells.

11. The method of claim 10, wherein the further transgene encodes an RNA that can reduce expression of an endogenous gene.

12. The method of claim 11, wherein the further transgene encodes an RNA for reducing expression of CCR5.

13. The method of claim 1, wherein the first and second resistance genes are a trimetrexate (TMTX) resistance gene and a hydroxyurea (HU) resistance gene, and the toxic compounds are TMTX and HU.

14. The method of claim 1, further comprising contacting the cell population with a cytotoxic compound for which there is not a resistance gene present in the cell population.

15. The method of claim 14, wherein the cytotoxic compound is Busulfan.

16. A composition comprising transgenic cells obtained by a method of claims 1-15, for use in the treatment of a subject.

17. A method of treating a subject having a disease comprising administering transgenic cells to the subject, wherein the cells are obtained by a method of claim 1.

18. A method for treating a disease in a subject comprising:
(a) obtaining a therapeutic cell population including cells comprising transgenes for expression of (i) a Trimetrexate (TMTX) resistance gene and (ii) a Hydroxyurea (HU) resistance gene; and
(b) administering the therapeutic cell population to a subject having a disease, wherein the cell population is enriched for cells comprising the transgenes by exposing the cell population to an effective amount of TMTX and HU, thereby selecting cells comprising the transgenes.

19. The method of claim 18, wherein exposing the cell population to an effective amount of TMTX and HU is before said administration step.

20. The method of claim 18, wherein exposing the cell population to an effective amount of TMTX and HU is after said administration step or before and after said administration step.

21. The method of claim 18, further comprising administering TMTX and HU to the subject in an amount effective to selectively enrich cells comprising the transgenes.

22. The method of claim 21, wherein the TMTX and HU are administered 2, 3, 4 or more times.

23. The method of claim 18, wherein exposing the cell population further comprises exposing the cell population to an effective amount of TMTX, HU and Busulfan.

24. The method of claim 18, wherein the cells comprise a further transgene.

25. The method of claim 24, wherein the TMTX resistance gene, the HU resistance gene and the further transgene are expressed a polycistronic transcript.

26. The method of claim 24, wherein the further transgene corrects a genetic defect in the cells of the subject.

27. The method of claim 24, wherein the further transgene encodes an RNA that can reduce expression of an endogenous gene.

28. The method of claim 24, wherein the further transgene encodes an RNA for reducing expression of CCR5.

29. The method of claim 18, wherein obtaining the cell population comprises transforming cells with at least a first vector comprising the transgenes.

30. The method of claim 29, wherein obtaining the cell population comprises transforming cells from the subject.

31. A method of treating an HIV infection in a subject comprising:
(a) obtaining a bone marrow cell population from the patient;
(b) transforming the cell population with one or more vectors comprising transgenes for expression of (i) a Trimetrexate (TMTX) resistance gene; (ii) a Hydroxyurea (HU) resistance gene; and (iii) a transgene that encodes an RNA for reducing expression of CCR5; and
(c) administering the transformed cell population to the subject,
wherein the cell population is enriched for cells comprising the transgenes by exposing the cell population to an effective amount of TMTX and HU, thereby selecting cells comprising the transgenes.

32. The method of claim 31, further comprising exposing the subject to radiation to ablate bone marrow cells after obtaining the bone marrow cell population from the subject.

33. The method of claim 31, further comprising administering TMTX and HU to the subject in an amount effective to selectively enrich cells comprising the transgenes.

34. A composition for use in the treatment of a disease comprising a cell population including cells comprising transgenes for expression of (i) a Trimetrexate (TMTX) resistance gene; and (ii) a Hydroxyurea (HU) resistance gene.

35. The composition of claim 34, wherein the cell population is a human cell population.

36. The composition of claim 35, wherein the cell population comprises primary cells.

37. The composition of claim 36, wherein the cell population comprises cells extracted from bone marrow.

38. The composition of claim 34, wherein the cell population comprises induced pluripotent cells.

39. The composition of claim 34, wherein the transgenes are comprised in an episomal vector or an integrated plasmid or a viral vector.

40. The composition of claim 39, wherein the viral vector is a lentiviral vector.

41. The composition of claim 34, wherein the cells comprise a further transgene.

42. The composition of claim 34, wherein the TMTX resistance gene, the HU resistance gene and the further transgene are expressed a polycistronic transcript.

43. The composition of claim 41, wherein the further transgene corrects a genetic defect in the cells.

44. The composition of claim 43, wherein the further transgene encodes an RNA that can reduce expression of an endogenous gene.

45. The composition of claim 44, wherein the further transgene encodes an RNA that can reduce expression of CCR5.

46. The composition of claim 45, further defined as a composition for use in treating HIV infection is a subject.

47. A recombinant polynucleotide molecule comprising a human RFC1 protein coding sequence and a human RRM2 protein coding sequence, wherein both protein coding sequences are operably linked to a promoter.

48. The polynucleotide of claim 47, wherein the molecule is comprises a retroviral vector.

49. The polynucleotide of claim 48, wherein the retroviral vector is a lentiviral vector.

50. The polynucleotide of claim 47, wherein the RFC1 protein coding sequence and the RRM2 protein coding sequence are operable linked to the same promoter.

51. The polynucleotide of claim 47, comprising a further transgene.

52. The polynucleotide of claim 51, wherein the further transgene encodes an RNA that can reduce expression of an endogenous gene.

53. The polynucleotide of claim 52, wherein the further transgene encodes an RNA for reducing expression of CCR5.

54. The polynucleotide of claim 51, wherein the further transgene is expressed a polycistronic transcript with the RFC1 protein coding sequence or the RRM2 protein coding sequence.

55. An in vitro method for selecting a transgenic cell comprising:
(a) obtaining a cell population including cells comprising transgenes for expression of (i) a trimetrexate (TMTX) resistance gene and (ii) a hydroxyurea (HU) resistance gene; and
(b) contacting the cell population with an effective amount of TMTX and HU, thereby selecting cells comprising the transgenes.

56. The method of claim 55, further comprising contacting the cell population with Busulfan.
